(19) United States
(12) Patent Application Publication
(10) Pub. No.: US 2022/0087609 A1
(43) Pub. Date: Mar. 24, 2022

(54) IN-EAR BIOMETRIC MONITORING USING PHOTOPLETHYSMOGRAPHY (PPG)

(71) Applicant: BOSE CORPORATION, Framingham, MA (US)

(72) Inventors: Tegan M. AYERS, Waltham, MA (US); Christopher R. PAETSCH, Cambridge, MA (US)

(21) Appl. No.: 17/420,341

(22) PCT Filed: Jan. 6, 2020
(86) PCT No.: PCT/US2020/012341

§ 371 Date: Jul. 1, 2021

Related U.S. Application Data
(60) Provisional application No. 62/789,345, filed on Jan. 7, 2019.

(51) Int. Cl. A61B 5/00; A61B 5/024; A61B 5/08; A61B 5/021; A61B 5/145

(52) U.S. Cl. CPC A61B 5/6817; A61B 5/4818; A61B 5/02405; A61B 5/02416; A61B 5/021; A61B 5/14542; A61B 5/14532; A61B 5/0816

(57) ABSTRACT

Aspects of the present disclosure provide methods and apparatuses for determining a subject's PPG based on a signal collected in-ear. Based on the PPG, one or more biometrics are determined including a heart rate, HRV, RR, SpO2 level, blood pressure, blood glucose level, or hemoglobin A1C level. RR and SpO2 are used to determine the presence of a sleep apnea event. The methods and apparatus of determining the subject's PPG described herein are continuous and non-invasive.

### CROSS-REFERENCE TO RELATED APPLICATIONS

This application claims priority to and benefit of U.S. Patent Application No. 62/789,345, filed Jan. 7, 2019, the contents of which are herein incorporated by reference in its entirety as fully set forth below.

### FIELD

Aspects of the present disclosure relate to determining or estimating a photoplethysmogram (PPG) of a subject based on signals received via a sensor on an in-ear audio device. As described herein, the PPG is used to determine or estimate a variety of biometric parameters. Any one of the determined biometric parameters are reported to the wearer or other individual, such as a physician, and/or used to create a closed-loop experience responsive to the determined parameter.

### BACKGROUND

Monitoring an individual's health helps in preventative care and with the diagnosis and treatment of diseases. PPG sensors optically detect changes in the blood flow volume (i.e., changes in the detected light intensity) in the microvascular bed of tissue. PPG is detected via reflection from or transmission through the tissue. PPG sensors can be used to estimate a variety of biometric parameters. Currently, clinical-grade fingertip or earlobe PPG sensors collect signals to calculate a subject's PPG. Fingertip and earlobe measures are currently preferred; however, these locations may have limited blood perfusion. In addition, conventional fingertip or earlobe PPG sensors may not be ideal for taking measurements over a long period of time. While these methods may be sufficient for hospital settings, methods and apparatus to monitor and calculate PPG in non-hospital settings are desired.

### SUMMARY

All examples and features mentioned herein can be combined in any technically possible manner.

According to aspects, an in-ear audio device is configured to determine, estimate, or calculate a subject's PPG. Specifically, at least one sensor disposed on an earpiece of the audio device is configured to collect signals of a subject wearing the audio device. The signals are used to determine or estimate the subject's PPG. In aspects, a processor onboard the audio device determines or estimates the PPG. In other aspects, an external device, coupled to the audio device, determines or estimates the subject's PPG. In other aspects, the subject's PPG is determined or estimated in the cloud.

Certain aspects provide a method for determining a photoplethysmogram (PPG) of a subject comprising receiving signals via a PPG sensor disposed on an in-ear audio device inserted in an ear of the subject, and taking one or more actions to estimate the subject's PPG based on the received signals.

In aspects, the one or more actions comprises transmitting, by the in-ear audio device, information associated with the received signals to a device external to the in-ear audio device and receiving, by the in-ear audio device, the estimate of the subject's PPG.

In aspects, the method further comprises estimating based, at least in part, on the subject's estimated PPG, one or more biometrics associated with the subject. In aspects, the one or more biometrics associated with the subject comprise at least one of: heart rate, heart rate variability (HRV), respiration rate (RR), peripheral capillary oxygen saturation (SpO2) level, blood pressure, blood glucose level, or hemoglobin A1C level.

In aspects the one or more biometrics associated with the subject comprise: respiration rate (RR) and peripheral capillary oxygen saturation (SpO2) level and the method further comprises detecting a sleep apnea event based on the RR and the SpO2 level.

In aspects, the PPG sensor comprises a plurality of transmitters and emitters, each disposed on an earbud of the in-ear audio device.

In aspects, receiving the signals via the PPG sensor comprises continuously receiving the signals over a sleep period. In aspects, the method further comprises determining a continuous blood pressure based on the estimated PPG.

In aspects, the method further comprises determining the subject is stressed based on the estimated PPG and adjusting an audio output in an effort to decrease at least one of the subject's respiration rate (RR), heart rate, heart rate variability (HRV), or blood pressure.

In aspects, the method further comprises performing closed-respiration entrainment based on a respiration rate (RR) determined using the estimated PPG.

Certain aspects provide an in-ear earpiece configured to determine a photoplethysmogram (PPG) of a subject comprising a PPG sensor configured to receive signals from an ear canal of a subject and at least one processor configured to take one or more actions to estimate the subject's PPG.

In aspects, the PPG sensor is disposed on an earbud of the in-ear earpiece. In aspects, the PPG sensor comprises a plurality of transmitters and emitters disposed on the in-ear earpiece.

In aspects, the in-ear earpiece further comprises a transceiver configured to transmit information associated with the received signals to an external device, and receive the estimate of the subject's PPG.

In aspects, the at least one processor is configured to estimate based, at least in part, on the subject's estimated PPG, one or more biometrics associated with the subject.

In aspects, the one or more biometrics associated with the subject comprise at least one of: heart rate, heart rate variability (HRV), respiration rate (RR), peripheral capillary oxygen saturation (SpO2) level, blood pressure, blood glucose level, or hemoglobin A1C level.

In aspects, the one or more biometrics associated with the subject comprise: respiration rate (RR) and peripheral capillary oxygen saturation (SpO2) level and the at least one processor is configured to detect a sleep apnea event based on the RR and the SpO2 level.

In aspects, the in-ear earpiece is part of a sleep mask.

Certain aspects provide a method for non-invasively determining at least one biometric parameter comprising measuring changes in blood volume within a blood vessel of a subject using a light emitting diode (LED) and photodetector (PD) disposed in on an ear tip of an in-ear earpiece, estimating a photoplethysmogram (PPG) of the subject based on the measured changes, estimating the at least one biometric parameter based on the estimated PPG, and taking one or more actions based on the at least one estimated biometric parameter.

In aspects, measuring the changes comprises continuously measuring the changes over a sleep period.

Advantages of determining a subject's PPG using signals collected in-ear will be apparent from the description and the claims.

### BRIEF DESCRIPTION OF THE DRAWINGS

FIG. 1 illustrates an example technique for using a fingertip sensor to determine a PPG.

FIG. 2 illustrates an example earpiece.

FIG. 3 illustrates example operations performed for determining a PPG by an in-ear audio device.

FIG. 4 illustrates an example PPG waveform.

FIG. 5 illustrates a PPG waveform correlating to a heart beats.

FIG. 6 illustrates a PPG waveform correlating to a respiration rate (RR).

FIG. 7 illustrates an example of deoxygenated hemoglobin and oxygenated hemoglobin absorbing red and infrared lights.

FIG. 8 illustrates an example of spontaneous breathing and sleep apnea events as shown in a PPG waveform.

FIGS. 9A-9D illustrate a correlation between spectral components of a blood pressure and PPG waveform.

### DETAILED DESCRIPTION

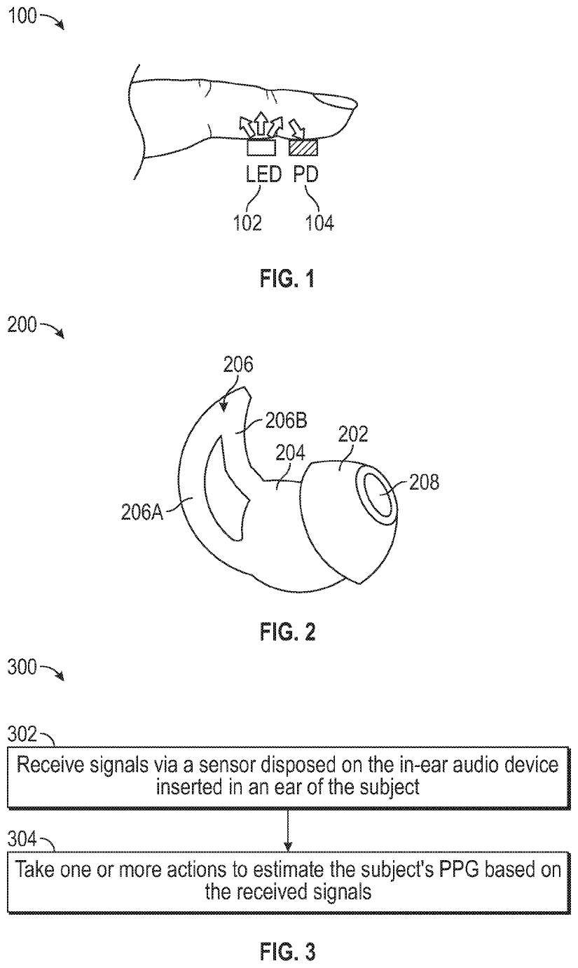

FIG. 1 illustrates an example technique 100 for using a fingertip sensor to determine a PPG. A PPG sensor measures changes in blood volume within a blood vessel using a light emitting diode (LED) 102 and photodetector (PD) 104. In an example, the LED 102 emits a light of a known wavelength, such as 640 nm (red) or 910 nm (infrared) through the soft tissue. The PD 104 detects light that is back-scattered or reflected from one or more of the tissue, bone, and/or blood vessels. The modulation of reflected light correlates to the volume of blood in the arteries.

A PPG can be used to detect a variety of biometric parameters, including, but not limited to, heart rate, heart rate variability (HRV), respiration rate (RR), peripheral capillary oxygen saturation (SpO2) level, blood pressure, cholesterol, heart disease, blood glucose, stress, and hemoglobin A1C level. One or more of these parameters, such as RR and SpO2, or other characteristics of the signal are used to determine a sleep apnea event. In aspects, a machine learning algorithm is used to determine a biometric parameter from the PPG signal obtained in-ear by an audio device.

While a fingertip or earlobe PPG sensor may be sufficient for a hospital setting, these locations do not allow for ambulatory measurements within the home or over long periods of time, such as while a subject is sleeping. Accordingly, aspects describe an in-ear PPG sensor configured to collect signals used to determine a subject's PPG regardless of the subject's location and over long periods of time, such as over several hours. According to aspects, an in-ear PPG sensor is used to non-invasively and continuously determine the subject's PPG. The ear, specifically the ear canal, provides a stable environment, which naturally occludes motion and ambient light artifacts, and contains an abundance of blood vessels. These factors contribute to a more accurate measurement of PPG, as compared to a wrist-worn PPG sensor. The ear canal also provides a location where PPG may be monitored over long periods of time. In aspects, the in-ear device monitors the subject's PPG over a sleep period, a wake period, or a combination of sleep and wake period, any of which can last several hours. In aspects, the in-ear device monitors the subject's PPG during periods of activity.

FIG. 2 illustrates an example in-ear earpiece. 200. The earpiece 200 includes a body 204 that houses an acoustic driver module, a nozzle 208 extending from the body 204 towards a substantially frusto-conical sealing structure 202, and a positioning and retaining structure 206.

The body 204 of the ear tip 200 is substantially elliptical or circular in shape. A substantially elliptical or circular shape is suited to align with typical ear geometries. The shape of the body 204 is configured to match the lower concha of a subject's ear. In an example, the body 204 houses an earbud including an acoustic driver module. The earbud may include a stem for positioning cabling and the like; however, some earbuds, and therefore earpieces, may lack the stem and may include electronic modules (not shown) for wirelessly communicating with external devices. Other earpieces may lack the stem and the acoustic driver module and may function as passive earplugs.

A nozzle 208 extends from the body 204 towards the ear canal of the subject's ear. The nozzle includes an acoustic passage to conduct sound waves to the ear canal of the subject.

The ear tip 202 provides enough surface area to contact the subject's skin when positioned in-ear. The soft, flexible material of the tip 202 helps the tip to conform to the subject's ear geometry and increases the surface area having contact with a subject's skin. Additionally, the flare of the umbrella shaped tip 202 provides some springiness so that some pressure is exerted by the tip 202 on the subject's skin.

The ear tip 202 includes at least one PPG sensor. In one aspect, the sensor includes an LED and a PD as shown in FIG. 1. In an example, the LED and PD are housed within the body, for example on or inside the nozzle of the earbud. When the sensor is contained within the nozzle, the light from the LED effectively passes through the tip 202 with little or no interference from the tip. In another example, the LED and PD are disposed on the back side of the umbrella tip, so that the light from the LED shines through the ear tip 202 and into the subject's ear canal. The PD measures the light that is reflected back. In another aspect, the ear tip includes multiple emitters and collectors. In an aspect, at least some of the multiple emitters and collectors are positioned on the back side of the umbrella tip. In an example, at least some of the collectors and emitters form a circular or semi-circular shape. In aspects, the PPG sensor, including one emitter and one collector, is disposed anywhere on the earpiece 200 where the PPG sensor is able to collect an in-ear PPG signal.

The positioning and retaining structure 206 holds the earpiece in position in a subject's ear, without significant contribution from the portions of the ear tip that engage the ear canal and without any structure external to the ear tip. In an example, as shown in FIG. 2, the ear tip 200 includes a positioning and retaining structure 206 having an outer leg 206A and an inner leg 206B; however, the disclosure is not limited to an ear tip having two legs. In an example, an ear tip includes a single leg extending from the body and configured to follow the curve of the anti-helix and/or the cymba concha at the rear of the concha.

In aspects, the earpiece 200 is connected, either via a wired connection or a wireless connection, to a second earpiece configured to fit in subject's right ear. In aspects, the earpiece is part of a wearable form factor, such as audio eyeglasses or a sleep mask.

In aspects, the earpiece 200 includes a PPG sensor, as described above, one or more of a memory and processor, communication unit, transceiver, and audio output transducer or speaker. In an aspect, any of the PPG sensor, memory and processor, communication unit, transceiver, and audio output transducer are configured to communicate with each other. In an example, all of these components are coupled to and communicate with each other.

The memory and processor control the operations of the earpiece 200. The memory stores program code for controlling the memory and processor. The memory may include Read Only Memory (ROM), a Random Access Memory (RAM), and/or a flash ROM. The processor controls the general operation of the earpiece 200. The processor performs process and control for audio and/or data communication. In some aspects, in addition to the general operation, the processor is configured to determine a subject's PPG and one or more biometric parameters associated with the subject based on the determined PPG. In aspects, the processor is configured to output the determined PPG and/or determined one or more biometric parameters determined from the PPG. In an example, the processor, in combination with one or more other components of the earpiece, perform the operations described with reference to FIG. 3.

The communication unit facilitates a wireless connection with one or more other devices. For example, the communication unit may include one or more wireless protocol engines such as a Bluetooth engine. While Bluetooth is used as an example protocol, other communication protocols may also be used. Some examples include Bluetooth Low Energy (BLE), Near Field Communications (NFC), IEEE 802.11, or other local area network (LAN) or personal area network (PAN) protocols.

In an example, the communication unit wirelessly communicates with an external device, such as a bedside unit, a tablet, a cell phone, a smart device, or the cloud. In an example, the communication unit wirelessly communicates an indication of the collected PPG waveform. Any of the external devices or cloud may determine the subject's PPG from the waveform and a biometric parameter based, at least in part, on the PPG.

The transceiver transmits and receives information via one or more antennae to exchange information with one or more other devices. The transceiver is not necessarily a distinct component. The audio output transducer may be also known as a driver or speaker. In some examples, more than one output transducer is used. The transducer converts electrical signals into sound and converts sound into electrical signals. In aspects, the transducer adjusts an audio output by the earpiece 200 based on a determined biometric parameter.

The earpiece 200 is provided for illustrative purposes only. Aspects of the disclosure are not limited to the specific form factor illustrated in FIG. 2 or described with reference to FIG. 2. According to aspects, any earpiece including a PPG sensor that contacts a subject's skin is configured to collect in-ear signals that are used to determine the subject's PPG.

FIG. 3 illustrates example operations performed for determining a PPG by an in-ear audio device. At 302, the in-ear audio device receives signals via a PPG sensor disposed on the in-ear audio device inserted in an ear of the subject. At 304, the audio device takes one or more actions to estimate the subject's PPG based on the received signals. In aspects, a PPG sensor is configured to receive signals from an ear canal of a subject.

In aspects, the one or more actions comprise transmitting, by the in-ear audio device, information associated with the received signals to a device external to the in-ear audio device and receiving, by the in-ear audio device, the estimate of the subject's PPG. In yet other aspects, the audio device itself determines the subject's PPG.

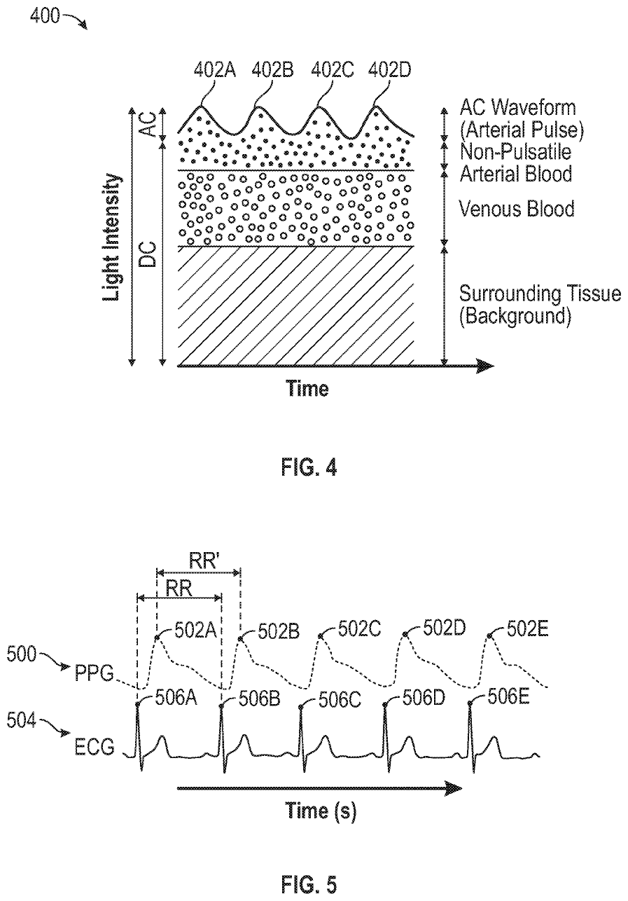

As described with reference to FIGS. 4-9, the audio device estimates one or more biometrics of the subject, which may be referred to as biometric parameters, based, at least in part, on the subject's estimated PPG. As described in more detail below, the one or more biometrics associated with the subject comprise at least one of: heart rate, heart rate variability (HRV), respiration rate (RR), peripheral capillary oxygen saturation (SpO2) level, blood pressure, blood glucose level, or hemoglobin A1C level. In aspects, a RR and SpO2 are estimated from a PPG signal collected in-ear. Based on the RR and SpO2 level, a sleep apnea event is detected.

In aspects, the PPG sensor comprises a plurality of transmitters and emitters, each disposed on an ear tip of the in-ear device. In aspects, one or more PPG sensors disposed on the in-ear device continuously receive the signals over a sleep period. Continuously receiving the signals over a period enables a non-invasive method for determining a continuous blood pressure based on the estimated PPG. The period of time may include several hours of sleeping, activity, or a combination of both. In an example, the subject's PPG is monitored overnight. In aspects, the PPG sensor measures changes in blood volume within a blood vessel of a subject using an LED and PD disposed on an ear tip of an in-ear earpiece.

In aspects, actions are taken based on the determined biometrics. The audio device may continuously calculate one or more of described biometrics based on a PPG signal collected in-ear. The audio device may continuously take action based on a determined state of the subject. For example, based on an elevated RR, heart rate, HRV, or blood pressure, the audio device may determine the subject is stressed. In response to determining the subject is stressed, the audio device adjusts an audio output to calm the subject in an effort to decrease the subject's RR, heart rate, HRV, or blood pressure. In another example, the audio device performs closed-loop respiration entrainment based on a RR determined using the estimated PPG. In closed-loop respiration entrainment, a subject is guided to a resting RR based on the subject's current RR. As used in herein, entrainment refers to guiding a user's respiration, breath, or breathing.

FIG. 4 illustrates an example PPG waveform 400. Peaks 402A-402D in the waveform 400 correspond to heart beats. Accordingly, a PPG signal is used to determine or monitor the subject's heart beats.

FIG. 5 illustrates a PPG waveform 500 correlating to a subject's heart beats. As noted above with respect to FIG. 4, peaks in the PPG waveform correlate to heart beats. Specifically, a high volume of blood within the vessels leads to increased light absorption and consequently, less light being reflected to the PD. To detect heart rate, the peaks 502A-502E of the time series PPG signal are identified. To determine an instantaneous heart rate, the peaks of the PPG signal are divided by the time period of observation. As shown in FIG. 5, a peak 502A-502E of the PPG signal 500 lags a corresponding peak 506A-506E of the ECG signal 504; however, the distance between consecutive ECG peaks (RR, where RR is the period between heart beats) is approximately equal to the distance between consecutive PPG peaks (RR').

HRV refers to the variation in time between heart beats. Identifying peaks in the PPG waveform, as shown above in FIGS. 4 and 5, and calculating a standard variation metric (e.g., standard deviation, root mean square of the successive difference, etc.) is indicative of the subject's HRV. Calculations performed within the frequency domain also produce accurate HRV detection (e.g., low frequency power, high frequency power, etc.). In an example, a time between peaks is determined and a standard deviation is performed. One of the low frequency or high frequency components of the PPG signal is used to determine the subject's HRV. In aspects, a machine learning algorithm may be used to determine the subject's HRV from a PPG signal.

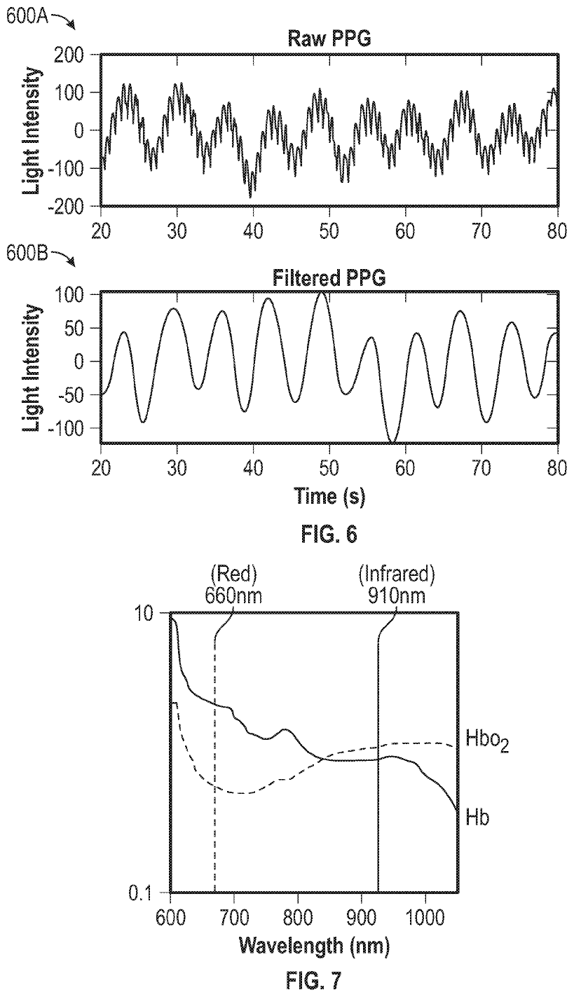

FIG. 6 illustrates a PPG waveform 600A used to determine a subject's respiration rate (RR). RR is included as low frequency content (e.g., 0.1-0.4 Hz) within the PPG signal. To extract a subject's RR, a raw PPG signal 600A is bandpass filtered, and the power spectral content is identified, for example, through performing a Fourier Transform on the signal. Peaks in the resulting filtered PPG signal 600B indicate the subject's RR. In aspects, a machine learning algorithm is used to determine the subject's RR from a PPG signal.

FIG. 7 illustrates an example of deoxygenated hemoglobin (Hb) and oxygenated hemoglobin (HbO2) absorbing red light and infrared light. SpO2 refers to the amount of oxygen present within the blood. A typical finger pulse oximeter, commonly used in a hospital setting, uses two LEDs of varying wavelengths, such as red (660 nm) and infrared (910 nm). Deoxygenated hemoglobin (Hb) will absorb more red light and oxygenated hemoglobin (HbO2) will absorb more infrared light. Measuring the light that is reflected back from the two LEDs and calculating a ratio of the measured light using the Formula 1 results in an R value, which correlates to a specific SpO2 value.

$$R \text{-} value correlates to SpO2 level \\ R value \approx \frac{\frac{{AC}_{red}}{{DC}_{red}}}{\frac{{AC}_{IR}}{{DC}_{IR}}} \qquad Formula 1$$

The intensity of light absorbed, and consequently, the intensity of light returned to the PD varies depending on which area of the body is measured. Therefore, measuring in-ear requires a novel SpO2 algorithm.

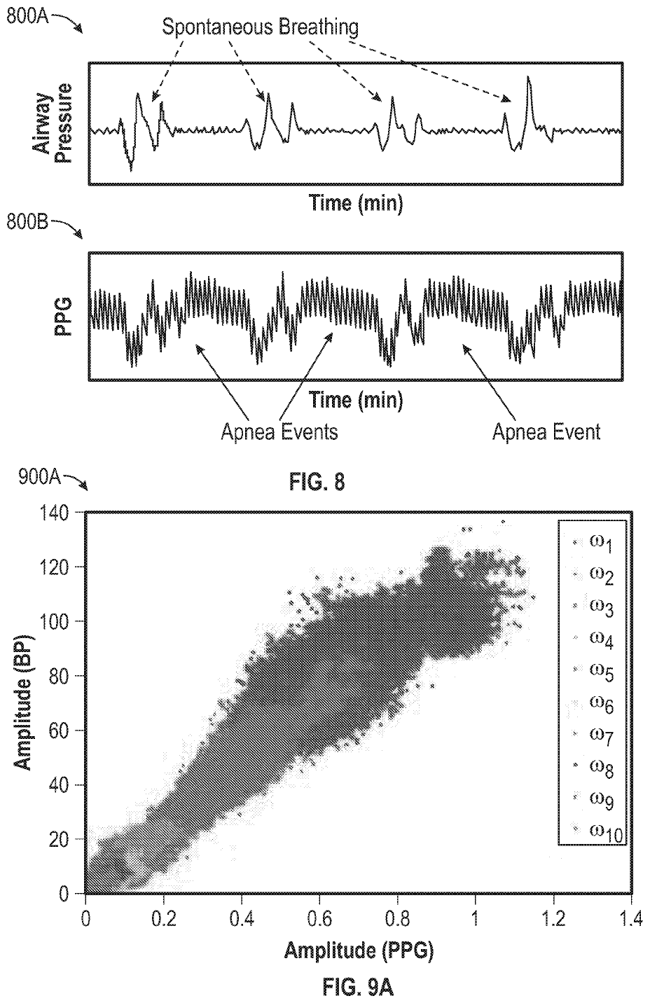

Sleep apnea is defined the cessation of breathing during sleep for greater than 10 seconds despite continual effort to breathe. During an apnea event, respiration ceases and SpO2 falls by greater than approximately 3%. As described above, respiration and SpO2 can be extracted from a PPG signal. Accordingly, using an in-ear PPG sensor, SpO2 and RR is estimated and used to identify a sleep apnea event. FIG. 8 illustrates an example waveform of spontaneous breathing 800A and sleep apnea events as shown in a PPG waveform 800B.

Continuous blood pressure is typically measured invasively, with electrodes directly placed on the artery. In contrast, a blood pressure cuff, which is considered non-invasive, does not allow for continuous measurements since it cuts off circulation to extremities during use. Continuous blood pressure is another biometric parameter that can be determined using a PPG signal obtained using an in-ear audio device. Specific characteristics of the PPG waveform, however, correlate to the arterial blood pressure waveform.

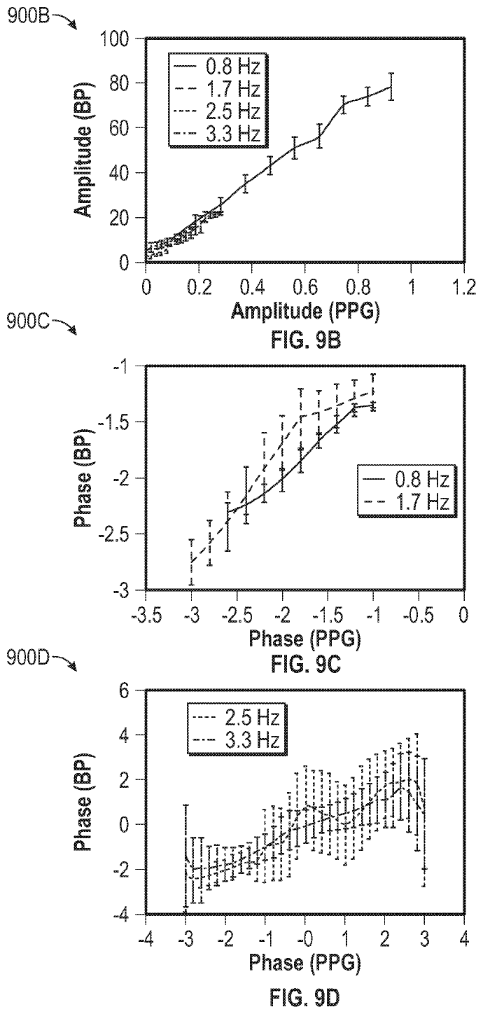

FIGS. 9A-9D illustrates a correlation between spectral components of a blood pressure and PPG waveform. FIG. 9A, 900A, and FIG. 9B, 900B, illustrate amplitude correlation between a blood pressure and PPG waveform and FIG. 9C, 900C, and FIG. 9D, 900D illustrate phase correlation between a blood pressure and PPG waveform. As shown in FIGS. 9A-9D, the amplitude (900A and 900B) and phase (900C and 900D) of a PPG signal is highly correlated to the arterial blood pressure signal. Therefore, an in-ear PPG sensor allows for continuous and non-invasive estimation of systolic and diastolic blood pressures.

Each biometric parameter described herein and sleep apnea are determined based on a PPG signal contained in-ear. Any combination of signal processing methods including filtering, smoothing, derivations, maxima, and minima and machine learning algorithms, such as recurrent or temporal convolutional neural networks are used to determine both the subject's PPG and the biometrics described herein. The methods described enable continuous, non-invasive PPG and biometric estimation. PPG and biometric estimation can be used to monitor a subject's health and identify sleep apnea events. Accordingly, in-ear PPG measurements are used to increase awareness about a subject's health in a non-invasive manner. Medical professionals or the subject may use this information to increase awareness about the subject's health and/or to address health concerns. In aspects, the PPG and estimated biometrics create closed-loop experiences in an effort to adjust one or more estimated biometric parameters.

In the preceding, reference is made to aspects presented in this disclosure. However, the scope of the present disclosure is not limited to specific described aspects. Aspects of the present disclosure may take the form of an entirely hardware embodiment, an entirely software embodiment (including firmware, resident software, micro-code, etc.) or an embodiment combining software and hardware aspects that may all generally be referred to herein as a “component,” “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium include: an electrical connection having one or more wires, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the current context, a computer readable storage medium may be any tangible medium that can contain or store a program.

The flowchart and block diagrams in the Figures illustrate the architecture, functionality and operation of possible implementations of systems, methods and computer program products according to various aspects. In this regard, each block in the flowchart or block diagrams may represent a module, segment or portion of code, which comprises one or more executable instructions for implementing the specified logical function(s). In some implementations, the functions noted in the block may occur out of the order noted in the figures. For example, two blocks shown in succession may, in fact, be executed substantially concurrently, or the blocks may sometimes be executed in the reverse order, depending upon the functionality involved. Each block of the block diagrams and/or flowchart illustrations, and combinations of blocks in the block diagrams and/or flowchart illustrations can be implemented by special-purpose hardware-based systems that perform the specified functions or acts, or combinations of special purpose hardware and computer instructions.

1. A method for determining a photoplethysmogram (PPG) of a subject comprising:
receiving signals via a PPG sensor disposed on an in-ear audio device inserted in an ear of the subject; and
taking one or more actions to estimate the subject's PPG based on the received signals.

2. The method of claim 1, wherein taking the one or more actions comprises:
transmitting, by the in-ear audio device, information associated with the received signals to a device external to the in-ear audio device; and
receiving, by the in-ear audio device, the estimate of the subject's PPG.

3. The method of claim 1, further comprising:
estimating based, at least in part, on the subject's estimated PPG, one or more biometrics associated with the subject.

4. The method of claim 3, wherein the one or more biometrics associated with the subject comprise at least one of: heart rate, heart rate variability (HRV), respiration rate (RR), peripheral capillary oxygen saturation (SpO2) level, blood pressure, blood glucose level, or hemoglobin A1C level.

5. The method of claim 3,
wherein the one or more biometrics associated with the subject comprise: respiration rate (RR) and peripheral capillary oxygen saturation (SpO2) level and further comprising:
detecting a sleep apnea event based on the RR and the SpO2 level.

6. The method of claim 1, wherein the PPG sensor comprises a plurality of transmitters and emitters, each disposed on an earbud of the in-ear audio device.

7. The method of claim 1, wherein receiving the signals via the PPG sensor comprises:
continuously receiving the signals over a sleep period.

8. The method of claim 7, further comprising:
determining a continuous blood pressure based on the estimated PPG.

9. The method of claim 1, further comprising:
determining the subject is stressed based on the estimated PPG; and
adjusting an audio output in an effort to decrease at least one of a respiration rate (RR), heart rate, heart rate variability (HRV), or blood pressure of the subject.

10. The method of claim 1, further comprising:
performing closed-respiration entrainment based on a respiration rate (RR) determined using the estimated PPG.

11. An in-ear earpiece configured to determine a photoplethysmogram (PPG) of a subject comprising:
a PPG sensor configured to receive signals from an ear canal of a subject;
at least one processor configured to take one or more actions to estimate the subject's PPG.

12. The in-ear earpiece of claim 11, wherein the PPG sensor is disposed on an earbud of the in-ear earpiece.

13. The in-ear earpiece of claim 11, wherein the PPG sensor comprises a plurality of transmitters and emitters disposed on the in-ear earpiece.

14. The in-ear earpiece of claim 11, further comprising:
a transceiver configured to:
transmit information associated with the received signals to an external device; and
receive the estimate of the subject's PPG.

15. The in-ear earpiece of claim 11, wherein the at least one processor is configured to estimate based, at least in part, on the subject's estimated PPG, one or more biometrics associated with the subject.

16. The in-ear earpiece of claim 15, wherein the one or more biometrics associated with the subject comprise at least one of: heart rate, heart rate variability (HRV), respiration rate (RR), peripheral capillary oxygen saturation (SpO2) level, blood pressure, blood glucose level, or hemoglobin A1C level.

17. The in-ear earpiece of claim 15,
wherein the one or more biometrics associated with the subject comprise: respiration rate (RR) and peripheral capillary oxygen saturation (SpO2) level; and
wherein the at least one processor is configured to detect a sleep apnea event based on the RR and the SpO2 level.

18. The in-ear earpiece of claim 11, wherein the in-ear earpiece is part of a sleep mask.

19. A method for non-invasively determining at least one biometric parameter comprising:
measuring changes in blood volume within a blood vessel of a subject using a light emitting diode (LED) and photodetector (PD) disposed in on an ear tip of an in-ear earpiece;
estimating a photoplethysmogram (PPG) of the subject based on the measured changes;
estimating the at least one biometric parameter based on the estimated PPG; and
taking one or more actions based on the at least one estimated biometric parameter.

20. The method of claim 19, wherein measuring the changes comprises:
continuously measuring the changes over a sleep period.
